(19) United States
(12) Patent Application Publication
(10) Pub. No.: US 2026/0294291 A1
(43) Pub. Date: Oct. 1, 2026

(54) MULTIDIMENSIONAL COMPENSATION AND IMPROVED R-VALUE ESTIMATION FOR PULSE OXIMETRY

(71) Applicant: Rajant Health Inc., Malvern, PA (US)

(72) Inventor: John Mountney, Malvern, PA (US)

(21) Appl. No.: 19/631,574

(22) Filed: Mar. 27, 2026

Related U.S. Application Data
(60) Provisional application No. 63/779,893, filed on Mar. 28, 2025.
(60) Provisional application No. 63/825,204, filed on Jun. 17, 2025.

(51) Int. Cl. A61B 5/1455; A61B 5/00; A61B 5/0533

(52) U.S. Cl. CPC A61B 5/14551; A61B 5/0533; A61B 5/6826; A61B 5/7267

(57) ABSTRACT

A pulse oximeter that improves the accuracy of blood oxygen saturation measurements by using skin moisture data (e.g., captured by a galvanic skin response or bioimpedance sensor) to account for variations in photoplethysmogram (PPG) caused by changes in the skin moisture level of the user. Additionally, in some embodiments, the pulse oximeter uses a modified Goertzel algorithm to calculate light absorption metrics (e.g., R-values) with higher accuracy and fewer computational resources than prior art devices. Additionally, in some embodiments, the pulse oximeter improves precision by calculating light absorption metrics at more frequency bands than the conventional ratio of red and infrared absorption.

### CROSS-REFERENCE TO RELATED APPLICATIONS

This application claims priority to U.S. Prov. Appl. No. 63/779,893, filed Mar. 28, 2025, and U.S. Prov. Appl. No. 63/825,204, filed Jun. 17, 2025, which are hereby incorporated by reference.

### BACKGROUND

Wearable medical devices (e.g., wristbands, fingertip sensors) typically use photoplethysmography (PPG) to measure blood oxygen saturation (SpO₂). A light emitting diode (LED) emits light, typically in the red and infrared spectrum, onto the skin. The light penetrates the skin and underlying tissues. As the light passes through the tissues, it is absorbed by blood and other components. Hemoglobin, the protein in red blood cells that carries oxygen, absorbs light differently depending on whether it is oxygenated or deoxygenated. A PPG sensor can either be a transmissive-type sensor or a reflective-type sensor. In a transmissive PPG sensor, a photodetector on the opposite side of the light source measures the amount of light that is transmitted through the tissues. In a reflective PPG sensor, a photodetector adjacent to the light source measures the amount of light that is reflected back from the tissues.

The light captured by the photodetector is converted into an electrical signal, which is then processed. Changes in light absorption during the cardiac cycle produce a waveform known as a photoplethysmogram (PPG). Because oxygenated and deoxygenated hemoglobin have different light absorption characteristics, a numerical model can be used to estimate the percentage of oxygen saturation in the blood based on the ratio of red-light absorption to infrared light absorption (referred to as the “R-value”).

Standard pulse oximeters typically use a single numerical model to estimate blood oxygen saturation based solely on the photoplethysmogram captured by the photodetector. However, because differences in skin tone (e.g., melanin levels) can cause variations in light absorption, scattering, and reflection, standard pulse oximeters can sometimes provide inaccurate readings. For individuals with darker skin, for instance, many existing pulse oximeters may underestimate oxygen saturation levels due to the way light interacts with melanin. Accordingly, Guo et al. (2023)¹ describe a pulse oximetry sensor that also measures the skin color of the user and selects one of a number of distinct numerical models, each calibrated for a distinct range of skin tones, to estimate the blood oxygen saturation level of the user. ¹ C.-Y. Guo, W.-Y. Huang, H.-C. Chang and T.-L. Hsieh, “Calibrating Oxygen Saturation Measurements for Different Skin Colors Using the Individual Typology Angle,” in IEEE Sensors Journal, vol. 23, no. 15, pp. 16993-17001, 1 Aug. 1, 2023, doi: 10.1109/JSEN.2023.3288151.

Some wearable health monitors (e.g., wrist worn devices such as smart watches and activity trackers) include a galvanic skin response (GSR) sensor that quantifies the skin moisture level of the user. A GSR sensor can be an impedance-based sensor or a capacitance-based sensor. Impedance-based GSR sensors use two electrodes placed on the skin of the user to apply a small AC current to the skin, measure the resulting voltage drop, and detect changes in impedance. Because changes in skin moisture level affect the conductivity (and, by extension, the impedance) of the skin, impedance-based GSR sensors can quantify the skin moisture level of the user based on those changes in impedance. Capacitance-based GSR sensors use two electrodes placed on the skin of the user to form a capacitor (with the skin acting as the dielectric material) and measure changes in the capacitance of the skin. Because changes in skin moisture affect the dielectric properties (and, by extension, the capacitance) of the skin, capacitance-based GSR sensors can quantify the skin moisture level of the user based on those changes in capacitance.

Additionally, or alternatively, the skin moisture level of a user may be quantified using a bioimpedance sensor. Impedance is a combination of resistance (opposition to current flow) and reactance (opposition due to capacitance, like cell membranes). Because water is a good conductor of electricity, skin with higher moisture content has a lower electrical impedance (i.e., it conducts electricity more easily) while drier skin has higher impedance. Accordingly, to estimate skin moisture level, a bioimpedance sensor may pass a small, safe AC current through electrodes placed on the skin. The applied AC current may be at a specific frequency (e.g., 3.3 kHz for skin moisture). The bioimpedance sensor measures the voltage drop across the electrodes and the current flowing through the skin. From those measurements, the device calculates the impedance of the skin. As with other biomarker estimations made based on sensor data, an algorithm is used to convert the calculated impedance of the skin into a quantifiable skin moisture level (e.g., in arbitrary units or as a percentage). Skin moisture level algorithms may also consider other skin parameters like oiliness and elasticity, as these can also influence impedance. While bipolar (two electrodes) measurements can be used, tetrapolar (four electrodes) configurations are often preferred to minimize errors caused by contact impedance between the electrodes and the skin.

Differences in skin moisture can also cause variations in light absorption, scattering, and reflection. However, prior art pulse oximeters-even those that quantify skin moisture level of the user-do not account for the variations caused by changes in skin moisture when estimating blood oxygen saturation. Therefore, variations in the skin moisture level of a user (e.g., caused by sweat or variations in hydration levels) can cause prior art pulse oximeters to provide inaccurate readings. Accordingly, there is a need for multidimensional compensation for pulse oximetry estimations.

Additionally, existing methods for determining R-values (namely, a fast Fourier transform (FFT) or the Goertzel algorithm) have significant drawbacks. A fast Fourier transform, for instance, is computationally expensive and will often inaccurately estimate both magnitude and phase without explicit knowledge of the target frequency. Meanwhile, both the FFT approach and the Goertzel algorithm are limited to integer frequency bins. Accordingly, there is a need for an improved method for estimating R-values based on GSR sensor data that reduces processing time and improves accuracy and precision.

### SUMMARY

In order to overcome those and other drawback of the prior art, a pulse oximeter is disclosed that improves the accuracy of blood oxygen saturation measurements by using skin moisture data (e.g., captured by a galvanic skin response or bioimpedance sensor) to account for variations in photoplethysmogram (PPG) caused by changes in the skin moisture level of the user. Additionally, in some embodiments, the pulse oximeter uses the modified Goertzel algorithm to calculate light absorption metrics (e.g., R-values) with higher accuracy and fewer computational resources than prior art devices. Additionally, in some embodiments, the pulse oximeter improves precision by calculating light absorption metrics at more frequency bands than the conventional ratio of red and infrared absorption.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a block diagram of a pulse oximeter that provides multidimensional compensation according to exemplary embodiments.

FIG. 2A is a diagram illustrating the pulse oximeter of FIG. 1 realized as a wearable health monitoring device according to exemplary embodiments.

FIG. 2B is another diagram illustrating the wearable health monitoring device of FIG. 2A according to exemplary embodiments.

FIG. 2C is a block diagram of the wearable health monitoring device of FIGS. 2A and 2B according to exemplary embodiments.

FIG. 3 is a flowchart illustrating a process for calculating light absorption metric(s) used to estimate the blood oxygen saturation (SpO2) of a user according to exemplary embodiments.

FIG. 4 is a diagram illustrating a neural network trained to learn a model for estimating the blood oxygen saturation (SpO2) of the user according to exemplary embodiments.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

### Multimodal Compensation for Pulse Oximetry

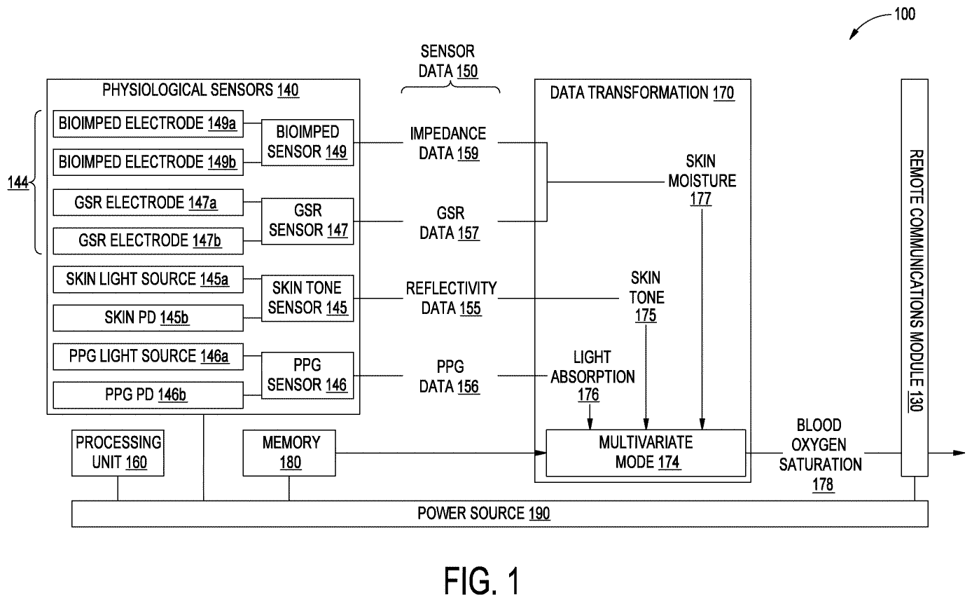

FIG. 1 is a block diagram of a pulse oximeter 100 that provides multidimensional compensation according to exemplary embodiments.

The pulse oximeter 100 includes physiological sensors 140, a remote communications module 130, a hardware processing unit 160, a memory 180, and a power source 190. In the embodiment of FIG. 1, the physiological sensors 140 include one or more skin moisture sensors 144, a photoplethysmography (PPG) sensor 146, and a skin tone sensor 145. In the embodiment of FIG. 1, the skin moisture sensors 144 include a galvanic skin response (GSR) sensor 147 and a bioimpedance sensor 149. In other embodiments, the pulse oximeter 100 may include only one skin moisture sensor 144. In those embodiments, the sole skin moisture sensor 144 may be, for example, a GSR sensor 147, a bioimpedance sensor 149, etc.

In the embodiment of FIG. 1, the bioimpedance sensor 149 includes two bioimpedance electrodes 149a and 149b and the GSR sensor 147 includes two GSR sensor electrodes 147a and 147b. In other embodiments, the bioimpedance sensor 149 and/or the GSR sensor 147 may include more electrodes (e.g., four electrodes). In the embodiment of FIG. 1, the PPG sensor 146 includes a PPG light source 146a and a PPG photodetector 146b and the skin tone sensor 145 includes a skin tone light source 145a and a skin tone photodetector 145b. In other embodiments, the PPG light source 146a and the skin tone light source 145a may be realized as a single light source. Additionally, or alternatively, the PPG photodetector 146b and the skin tone photodetector 145b may be realized as a single photodetector. Alternatively, the skin tone sensor 145 and the PPG sensor 146 may be realized as a single device.

Each of the physiological sensors 140 outputs sensor data 150 to a data transformation module 170, which processes that sensor data 150 to estimate the blood oxygen saturation 178 of the user. Specifically, the bioimpedance sensor 149 outputs impedance data 159 indicative of the impedance of the skin of the user, the GSR sensor 147 outputs GSR data 157 indicative of the galvanic skin response of the user, the PPG sensor 146 outputs PPG data 156 indicative of a photoplethysmogram of the user, and the skin tone sensor 145 outputs reflectivity data 155 indicative of the skin tone of the user. The data transformation module 170 may be realized as software instructions stored in the memory 180 and executed by the processing unit 160. Additionally, or alternatively, some of all of the physiological sensors 140 may be configured to perform one or more functions ascribed to the data transformation module 170.

Unlike conventional pulse oximeters that estimate blood oxygen saturation 178 based solely on PPG data 156, the disclosed pulse oximeter 100 uses multidimensional compensation to account for variance in the PPG data 156 that is unrelated to the blood oxygen saturation 178 of the user. Specifically, the disclosed pulse oximeter 100 uses information indicative of the skin moisture of the user (e.g., the GSR data output by the GSR sensor 147 and/or the impedance data 159 output by the bioimpedance sensor 149) to account for variations in the PPG data 156 caused by changes in the skin moisture level of the user described above. Additionally, in the embodiment of FIG. 1, the pulse oximeter 100 uses the reflectivity data 155 output by the skin tone sensor 145 to account for the skin tone of the user (and its effect on the PPG data 156 described above).

The data transformation module 170 uses the GSR data 157 output by the GSR sensor 147 and/or the impedance data 159 output by the bioimpedance sensor 149 to quantify the skin moisture level 177 of the user; uses the reflectivity data 155 output by the skin tone sensor 145 to quantify the skin tone 175 of the user; and uses the PPG data 156 output by the PPG sensor 146 to calculate one or more light absorption metrics 176 indicative of the light absorbed by the user. The light absorption metric(s) 176 may be the ratio of red light absorption to infrared light absorption (R-value) or a number of advanced absorption metrics 176 (or a composite absorption metric 176 calculated based on those advanced absorption metrics 176) as described in more detail below. To estimate the blood oxygen saturation 178 of the user, the skin moisture level 177 and skin tone 175 of the user are provided along with the light absorption metric(s) 176 to a multivariate model 174 stored in the memory 180. The multivariate model 174 may be, for example, a linear regression model realized as a formula

$$Ax + By + Cz + D = {SpO}_{2} \qquad [ Eq . 1 ]$$

where x is the light absorption metric 176, y is the skin tone 175 of the user, z is the skin moisture level 177 of the user, SpO₂ is the estimated blood oxygen saturation 178 of the user, A is a constant indicative of the correlation (in this example, a linear correlation) between the PPG data 156 and the estimated blood oxygen saturation 178, B is a constant indicative of the correlation (in this example, a linear correlation) between the skin tone 175 of the user and the estimated blood oxygen saturation 178, C is a constant indicative of the correlation (in this example, a linear correlation) between the skin moisture level 177 of the user and the estimated blood oxygen saturation 178, and D is an offset.

The multivariate model 174 may be generated by a neural network that is trained using data from a clinical trial (e.g., a hypoxia study) to generate a mathematical model for estimating blood oxygen saturation 178 based on light absorption metric(s) 176, skin tone 175, and skin moisture level 177. During the clinical trial, data is gathered from participants, including blood oxygen saturation 178, light absorption metric(s) 176, skin tone 175, and skin moisture level 177. That data is provided to a neural network that may include an input layer that takes the inputs (the light absorption metric(s) 176, the skin tone 175, and the skin moisture level 177), hidden layers with neurons that apply activation functions (e.g., ReLU) to capture complex, non-linear relationships between those inputs and the output, and an output layer that outputs an estimated blood oxygen saturation 178.

The dataset from the clinical trial is split into training data used to train the neural network and a validation set used to monitor the performance of the multivariate model 174 and prevent overfitting. A loss function (e.g., mean squared error) is used to measure the difference between the predicted blood oxygen saturation 178 output by the multivariate model 174 and the actual blood oxygen saturation 178 values captured during the clinical trial and an optimizer (e.g., Adam, stochastic gradient descent, etc.) adjusts the weights of the network (e.g., the coefficients A, B, C, and the offset D above) to minimize the loss function.

Once the neural network is trained, the multivariate model 174 generated by the neural network is used by the disclosed pulse oximeter 100 to estimate the blood oxygen saturation 178 of the user. Unlike conventional pulse oximeters that estimate blood oxygen saturation 178 based solely on PPG data 156, the disclosed pulse oximeter 100 accounts for the skin tone 175 of the user (and its effect on the PPG data 156) and variations in the PPG data 156 caused by changes in the skin moisture level 177 of the user.

Depending on the relationships between the inputs and the output, the neural network may generate a linear multivariate model 174 as described above or a non-linear multivariate model 174. The multivariate model 174 may be realized as a formula stored in the memory 180 and executed by the processing unit 160 as described above. Alternatively, the multivariate model 174 may be realized as one or more look-up tables stored in the memory 180 and accessed by the processing unit 160. The pulse oximeter 100 may use a single multivariate model 174 that takes the inputs described above (the one or more light absorption metrics 176, the skin tone 175, and the skin moisture level 177). Alternatively, the pulse oximeter 100 may use multiple models that are each calibrated for one or more of those inputs. For instance, the pulse oximeter 100 may store multiple models, which are each calibrated for a distinct range of skin tone values 175, that estimate blood oxygen saturation 178 based on the light absorption metric(s) 176 and skin moisture level 177.

The skin tone sensor 145 may be realized as any hardware device suitably capable of capturing reflectivity data 155 indicative of the skin tone 175 of the user. For example, the skin tone sensor 145 may be realized as an AMS OSRAM TCS3701 color and proximity sensor, which provides ambient light and color (RGB) sensing in parallel with IR proximity detection. The data transformation module 170 may use the reflectivity data 155 to quantify the user skin tone 175, for example, by calculating the individual typology angle (ITA) quantization value as described in Guo et al. (2023).

The PPG sensor 146 may be any device suitably configured to capture a photoplethysmogram (PPG) indicative of light absorbed by the user. For example, the PPG sensor 146 may be a transmissive PPG sensor (with the PPG photodetector 146b on the opposite side of the PPG light source 146a) that measures the amount of light that is transmitted through the tissues of the user or a reflective PPG sensor (with the PPG photodetector 146b adjacent to the PPG light source 146a) that measures the amount of light that is reflected back from the tissues of the user.

The GSR sensor 147 may be any device suitably configured to capture data indicative of the galvanic skin response of the user. For example, the GSR sensor 147 may be an impedance-based sensor (that uses the two GSR sensor electrodes 147a and 147b to apply a small AC current to the skin of the user, measure the resulting voltage drop, and detect changes in impedance) or a capacitance-based sensor that uses the two GSR sensor electrodes 147a and 147b to form a capacitor (with the skin of the user acting as the dielectric material) and measures changes in the capacitance of the skin.

The disclosed pulse oximeter 100 may be realized as a fingertip sensor, a wristband, etc. The memory 180 may include any non-transitory computer readable storage media (e.g., a hard drive, flash memory, etc.). The power source 190 may include any device suitably capable of providing power to the pulse oximeter. For example, the power source 190 may be a rechargeable battery, an energy harvesting mechanism that harvests energy (e.g., from body heat, motion, etc.), etc.

The processing unit 160 may include any number of hardware computing devices that are suitably adapted to perform the functions described herein. For example, the processing unit 160 may include a microprocessor, a central processing unit, and/or a microcontroller that performs some or all of those functions by executing software instructions stored in the memory 180 and/or an application specific integrated circuit (ASIC) or a field programmable gate array (FPGA) having hardware elements manufactured or configured to perform some or all of those functions. While the processing unit 160 is shown and described as a single component that is separate from each of the physiological sensors 140, those of ordinary skill in the art will recognize that some or all of the physiological sensors 140 may include a hardware processing unit adapted to perform some of the calculations ascribed herein to the processing unit 160. Accordingly, as used herein, the processing unit 160 includes any number of hardware devices whether separate from integrated with any of the physiological sensors 140.

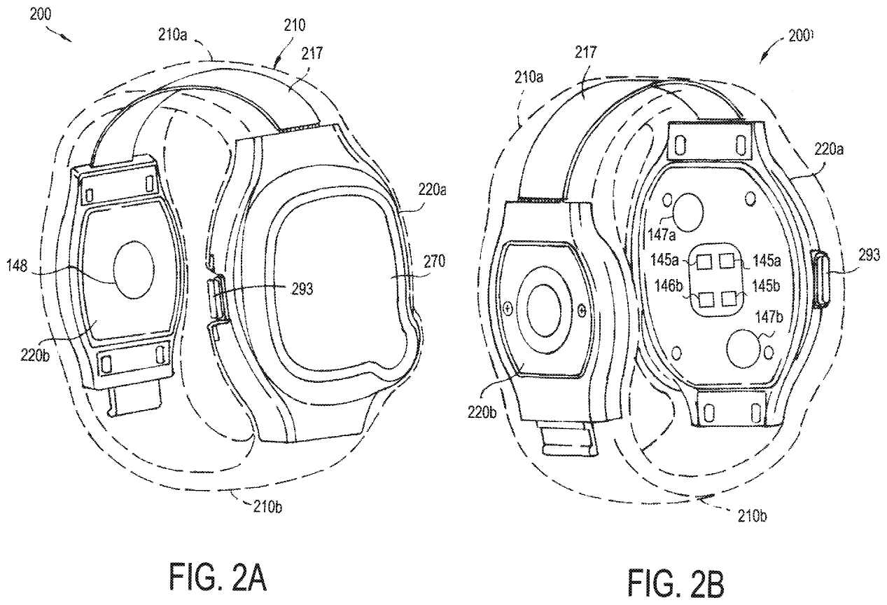

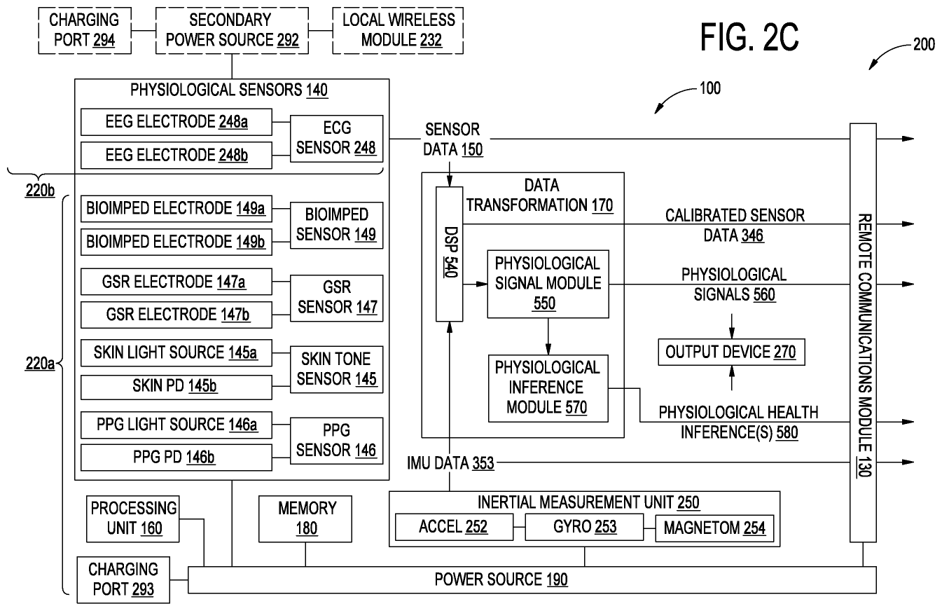

FIGS. 2A and 2B illustrate the disclosed pulse oximeter 100 realized as a wearable health monitoring device 200 (e.g., a modular wristband and sensing system as described in U.S. patent application Ser. No. 17/806,477, filed Jun. 10, 2022, the disclosure of which is hereby incorporated by reference).

In the embodiments of FIGS. 2A-2B, the wearable health monitoring device 200 includes two sensor modules 220a and 220b connected to wristband segments 210a and 210b to form a wristband 210. The sensor module 220a includes an output device 270 (in this embodiment, a display). In the embodiment of FIGS. 2A-2B, the sensor module 220a includes a charging port 293 for charging the power source 190 (in this example, a rechargeable battery) that provides power to the sensor module 220a and the sensor module 220b via wiring 217 (e.g., flex circuitry) in the wristband 210. Other embodiments may not include wiring 217. Instead, in those embodiments, the sensor module 220b may wirelessly communicate with the sensor module 220a via a direct, short range communication protocol (e.g., Zigbee, Bluetooth, etc.) and may include a battery and a charging port for providing power to the battery (as described below with reference to FIG. 2C).

The wearable health monitoring device 200 may include any of a number of physiological sensors 140. In the embodiment of FIGS. 2A-2B, the sensor module 220a includes the bioimpedance sensor 149 (having the bioimpedance electrodes 149a and 149b), the GSR sensor 147 (having the GSR sensor electrodes 147a and 147b), the PPG sensor 146 (having the PPG light source 146a and the PPG photodetector 146b), and the skin tone sensor 145 (having the skin tone light source 145a and the skin tone photodetector 145b). As mentioned above, in other embodiments, the PPG light source 146a and the skin tone light source 145a may be realized as a single light source, the PPG photodetector 146b and the skin tone photodetector 145b may be realized as a single photodetector, and/or the PPG sensor 146 and the skin tone sensor 145 may be realized as a single device.

The wearable health monitoring device 200 may also include additional physiological sensors 140 used to determine physiological metrics that are unrelated to blood oxygen saturation 178. In the embodiment of FIGS. 2A-2B, for example, the sensor module 220b includes an ECG sensor 248 having ECG sensor electrodes 248a and 248b shown in FIG. 2C and described below.

FIG. 2C is a block diagram of the wearable health monitoring device 200 according to exemplary embodiments.

In the embodiment of FIG. 2C, the wearable health monitoring device 200 includes two sensor modules 220a and 220b, each with one or more physiological sensors 140. The wearable health monitoring device 200 also includes a remote communications module 230, an inertial measurement unit 250, the hardware computer processing unit 160, the memory 180, the power source 190, a charging port 293, output device(s) 270, and the data transformation modules 170.

The remote communications module 230 enables the wearable health monitoring device 200 to output data for transmittal to a local computing device. The remote communications module 230 may include, for example, a module for short range, direct, wireless communication (e.g., Bluetooth, Zigbee, etc.) and/or a module for communicating via a local area network (e.g., WiFi). In other embodiments, the remote communications module 230 may enable the wearable health monitoring device 200 to bidirectionally communicate with a server via the one or more communications networks (e.g., cellular networks, the internet, etc.).

The output device 270 may include a display (e.g., as shown in FIGS. 2A-2B), a speaker, a haptic feedback device, etc. The power source 190 provides power to the sensor module 220a. In some embodiments, the power source 190 also provides power to the sensor module 220b via the wire 217 described above. In those embodiments, the sensor module 220b transfers data (e.g., output by the ECG sensor 248) to the sensor module 220a via the wire 217. In other embodiments, however, the sensor module 220b wirelessly communicates with the sensor module 220a via a direct, short range communication protocol (e.g., Zigbee, Bluetooth, etc.). In those embodiments, the sensor module 220b may also include a local wireless module 232 for sending data to the sensor module 220a. Additionally, in embodiments where power is not transmitted through the wiring 217, the sensor module 220b may include a secondary power source 292 (such as a battery and charging port 294 for providing power to the secondary power source 292). The charging port 293 (and the charging port 294) may be hardware ports for receiving electrical power (e.g., a universal serial bus port, an inductive charging port, etc.)

The physiological sensors 140 may include any device capable of sensing data indicative of a physiological or biochemical condition of the wearer.

The inertial measurement unit 250 may be any device capable of measuring and reporting the specific force and angular rate of the wearable health monitoring device 200. The inertial measurement unit 250 may also measure and report the orientation of the wearable health monitoring device 200. In the embodiment of FIG. 2C, the inertial measurement unit 250 includes an accelerometer 252 (e.g., a 3-axis accelerometer), a gyroscope 253, and a magnetometer 254. The inertial measurement unit 250 outputs IMU data 353 indicative of the movement of the wearable health monitoring device 200.

As described in U.S. patent application Ser. No. 17/806,477, the data transformation module 170 may use the IMU data 353 and digital signal processing 540 to calibrate the sensor data 150 (e.g., to remove motion artifacts and/or noise) and form calibrated sensor data 346. The data transformation module 170 may also include a physiological signal module 550 that identifies physiological signals 560 (e.g., the blood oxygen saturation 178 of the user, estimated as described above) based on the calibrated sensor data 346, and a physiological inference module 570 that makes physiological health inferences 580 based on those physiological signals 560 (e.g., an alert if the blood oxygen saturation 178 of the user drops below a threshold).

The remote communications module 230 outputs the sensor data 150, the calibrated sensor data 346, the physiological signals 560, and/or any physiological health inferences 580 for transmittal to a local computing device and/or a server. (The remote communications module 230 may also output the IMU data 353 for remote calibration of the sensor data 150.) In some embodiments, the physiological signals 560 may also be output to the user via an output device 270 (e.g., displayed to the user via a display). Physiological health inferences 580 may also be output to the user via an output device 270. For example, a visual, audible, and/or tactile alert may be output to the user via a display, a speaker, and/or a haptic feedback device.

### Improved Light Absorption Quantification

When calculating blood oxygen saturation (SpO₂) 178 using photo-plethysmography PPG signals 156, traditional methods compute the R-value from light absorption at two wavelengths λ₁ and λ₂ (typically red and infrared) using the ratio-of-ratios method. The process relies on the fact that the absorption ratio of pulsatile signals AC to baseline signals DC is related to blood oxygen saturation (SpO₂) 178 through the Beer-Lambert Law. The R-value is traditionally calculated as

$$R = \frac{\frac{{AC}_{{λ}_{1}}}{{DC}_{{λ}_{1}}}}{\frac{{AC}_{{λ}_{2}}}{{DC}_{{λ}_{2}}}} \qquad [ Eq . 2 ]$$

The computed R-value is then traditionally used as the input argument of a first or second order polynomial. Blood oxygen saturation (SpO₂) 178 is then estimated as

$${SpO}_{2} = {c}_{2} {R}^{2} + {c}_{1} R + {c}_{0} \qquad [ Eq . 3 ]$$

where the polynomial coefficients c₀, c₁, etc. are derived, for example, from empirical data collected during a hypoxia study.

The AC and DC components of the red and infrared PPG signals 156 are typically computed using a fast Fourier transform FFT, where the DC component corresponds with frequency bin zero and the frequency bin with the greatest magnitude is assumed to contain the AC term. That FFT approach, however, has a number of technical drawbacks. First, the FFT is computationally expensive. Additionally, without explicit knowledge of the target frequency, both the magnitude and phase estimation are often inaccurate.

Alternatively, the Goertzel algorithm offers a highly efficient digital signal processing algorithm used to calculate the discrete Fourier transform (DFT) at specific frequencies, particularly in applications where only a few frequency bins (or points) are of interest. It is often used in scenarios where it is desirable to detect a particular frequency or a set of frequencies, and the full spectrum is not necessary, which makes it much more computationally efficient than the standard FFT for such tasks. However, the Goertzel algorithm is also limited to integer frequency bins just as is the FFT approach.

An enhancement of the original Goertzel algorithm is the modified Goertzel. The modified Goertzel filter is a variation of the Goertzel algorithm that allows the DFT to be calculated at fractional frequency bins, not just integer frequency bins. That is useful when more precise frequency resolution is needed at frequencies that are not aligned with integer bins. The modified Goertzel results in more accurate AC and DC estimates of a periodic signal while simultaneously exploiting the computational efficiencies of the original algorithm. In order to effectively utilize that methodology, the pulsatile frequency f₀ of the PPG signal 156 must be known a priori.

Accordingly, when calculating the light absorption metrics (176) used to estimate blood oxygen saturation (SpO₂) 178 (for example, using information from the wearable health monitoring device 200), the disclosed pulse oximeter 100 may use the modified Goertzel for magnitude and phase estimation as described below.

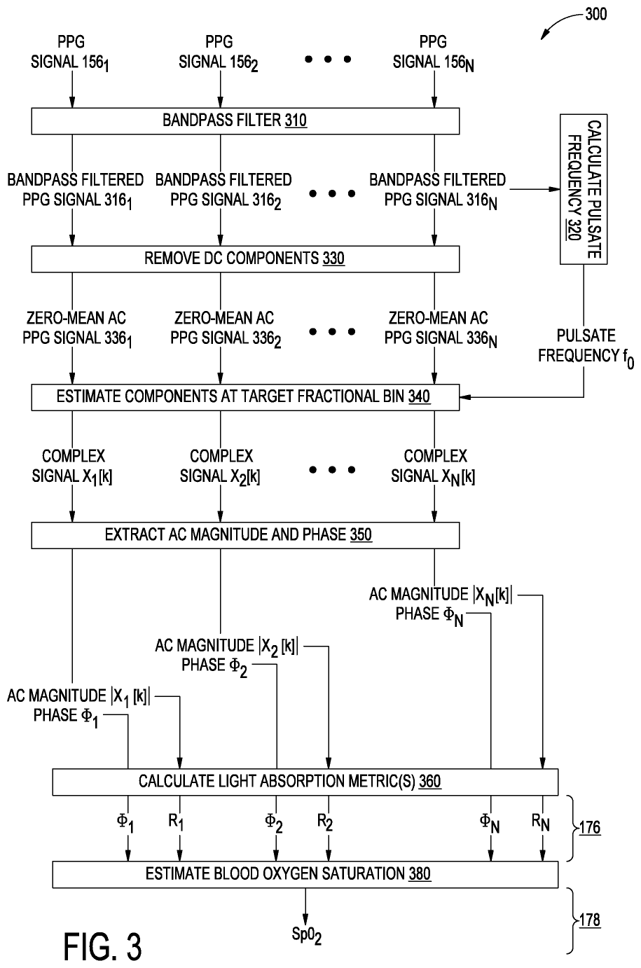

FIG. 3 is a flowchart illustrating a process 300 for using the modified Goertzel to calculate the one or more light absorption metrics 176 used to estimate blood oxygen saturation (SpO₂) 178 according to exemplary embodiments. The process 300 may be executed by the data transformation module 170 of the pulse oximeter 100 (e.g., a physiological inference module 570 of the wearable health monitoring device 200), which may be realized as software instructions stored in the memory 180 and executed by the processing unit 160 of the pulse oximeter 100 (e.g., wearable health monitoring device 200).

Each PPG signal 156ₙ at each of N wavelengths in is bandpass filtered in step 310, for example using zero-phase filtering or an infinite impulse response (IIR) filter, to form a bandpass filtered PPG signal 316ₙ.

In a zero-phase filtering process, the input PPG signal 156ₙ at each wavelength λₙ is filtered once forward in time to introduce a phase distortion. The output of the forward pass is then flipped in time and the flipped signal is passed through the same filter again, which introduces an equal and opposite phase distortion. The forward and reverse filtered signal is then flipped back to its original order, forming a zero-phase filtered signal in which backward-pass phase distortion cancels out the forward-pass distortion.

Alternatively, because zero-phase filtering is computationally expensive (and power and resources are scarce in embedded systems like the wearable health monitoring device 200), the pulse oximeter 100 may bandpass filter each PPG signal 156ₙ using an IIR bandpass filter that processes the PPG data 156ₙ sample-by-sample in the time domain. While an IIR bandpass filter introduces a slight time lag relative to a zero-phase filter, an IIR bandpass filter achieves nearly the same result as a zero-phase filter while using less power and fewer computational resources.

The pulsatile frequency f₀ of the bandpass filtered PPG signals 316 is estimated in step 320, for example using a single bandpass filtered PPG signal 316ₙ at a single selected wavelength λₙ. The bandpass filtered PPG signals 316 at every wavelength have the same observable pulsatile frequency f₀ induced by blood flow. Meanwhile, green light is less sensitive to noise from motion artifacts and other physiological sources compared to other wavelengths (e.g., red and infrared). Therefore, in some embodiments, the bandpass filtered PPG signal 316ₙ at a green wavelength may be used to provide a more accurate estimate of the fundamental frequency f₀ compared to bandpass filtered PPG signals 316 at red or infrared wavelengths. (In other embodiments, the pulsatile frequency f₀ may be estimated using a combination or fusion of bandpass filtered PPG signals 316 across multiple wavelength channels.)

In some embodiments, the pulsatile frequency f₀ may be estimated using a peak detection algorithm. In those embodiments, the bandpass filtered PPG signal 316ₙ (e.g., at the selected wavelength in) may be normalized between 0 and 1 and a cubic spline interpolation may be performed about each peak index to obtain finer time resolution of the true peak location. Locations of troughs in the bandpass filtered PPG signal 316, may then be identified by inverting the normalized signal and processing it through the same peak detection algorithm. Again, peaks (troughs) may be identified and a spline interpolation may be performed to obtain more accurate estimates of the true trough locations. The PPG pulsatile frequency f₀ may then be estimated as follows:

$${f}_{peaks} = \frac{{f}_{s}}{{\overline{Δ t}}_{peak}} \qquad [ Eq . 4 ]$$

$${f}_{troughs} = \frac{{f}_{s}}{{\overline{Δ t}}_{trough}} \qquad [ Eq . 5 ]$$

$${f}_{0} = \frac{{f}_{peaks} + {f}_{troughs}}{2} \qquad [ Eq . 6 ]$$

where fₛ is the sampling rate, Δ̅t̅ₚₑₐₖ is the average time between peaks, and Δ̅t̅_{trough} is the average time between troughs.

In other embodiments, the pulsatile frequency f₀ may be estimated using another method, such as autocorrelation, wavelets, zero-crossing, or a machine learning or other artificial intelligence model.

The DC component of each bandpass-filtered PPG signal 316ₙ at each wavelength λₙ is removed in step 330 to form a zero-mean AC PPG signal 336ₙ. For each bandpass-filtered PPG signal 316ₙ at each wavelength λₙ, for example, the DC component may be calculated using the standard Goertzel algorithm evaluated at bin k=0, which is equivalent to computing the zeroth bin of the Discrete Fourier Transform (DFT) (i.e., the mean value of the bandpass-filtered PPG signal 316ₙ). The DC component at each wavelength in is then subtracted from the bandpass-filtered PPG signal 316ₙ at the respective wavelength λₙ.

The AC component Xₙ[k] of each zero-mean AC PPG signal 336ₙ at each wavelength λₙ is calculated at the target fractional bin k in step 340, for example using the modified Goertzel. For a PPG sequence of length T, for instance, the target frequency bin k for the modified Goertzel is calculated as a function of the pulsatile frequency f₀ estimated in step 320 and the sampling rate fₛ:

$$k = \frac{{f}_{0} T}{{f}_{s}} \qquad [ Eq . 7 ]$$

The magnitude |Xₙ[k]| and the phase φₙ of the AC component Xₙ[k] at wavelengths λₙ are estimated in step 350 using the resulting complex-valued output Xₙ[k] from the modified Goertzel.

$$\vert {X}_{n} [ k ] \vert = \sqrt{{\text{?}}_{n}^{2} + {\text{?}}_{n}^{2}} \qquad [ Eq . 8 ]$$

$${ϕ}_{n} = {tan}^{- 1} \frac{{\text{?}}_{n} ( {X}_{n} [ k ] )}{{\text{?}}_{n} ( {X}_{n} [ k ] )} \qquad [ Eq . 9 ]$$

One or more light absorption metrics 176 are calculated in step 360.

In some embodiments, the light absorption metric 176 may be the R-value calculated based on the PPG signals 156 at two wavelengths two wavelengths λ₁ and λ₂ using Equation 2 above. In other embodiments, an improved light absorption metric 176 may be used that captures phase changes at two wavelengths λ₁ and λ₂ as blood oxygen saturation (SpO₂) 178 varies. In those embodiments, the improved light absorption metric 176 (R′) may be calculated using a model that integrates the traditional elements of Equation 2 with the phase information obtained from Equation 9, for example as shown in Equation 10:

$${R}^{′} = \frac{\frac{{AC}_{{λ}_{1}}}{{DC}_{{λ}_{1}}} + αcos ( Δϕ )}{\frac{{AC}_{{λ}_{2}}}{{DC}_{{λ}_{2}}} + βcos ( Δϕ )} \qquad [ Eq . 10 ]$$

where Δφ is the difference between φ₁ and φ₂ and the phase difference coefficients α and β is derived from empirical data collected during a hypoxia study as described above.

In other embodiments, the improved light absorption metric 176 (R′) may be based on light absorption at more than two wavelengths λₙ, for example based on a linear combination of wavelength-specific light absorption metric 176 (Rₙ):

$${R}^{′} = {β}_{0} + {β}_{1} {R}_{1} + {β}_{2} {R}_{2} + … + {β}_{N} {R}_{N} \qquad [ Eq . 11 ]$$

where each wavelength-specific light absorption metric 176 (Rₙ) is the ratio of pulsatile AC components to the non-pulsatile DC components at each λₙ:

$${R}_{1} = \frac{{AC}_{{λ}_{1}}}{{DC}_{{λ}_{1}}} \qquad [ Eq . 12 ]$$

$${R}_{2} = \frac{{AC}_{{λ}_{2}}}{{DC}_{{λ}_{2}}}$$

$$…$$

$${R}_{N} = \frac{{AC}_{{λ}_{N}}}{{DC}_{{λ}_{N}}}$$

and the light absorption coefficients β₀, β₁, . . . β_{N} are derived from empirical data collected during a hypoxia study as described above.

In other embodiments, each wavelength-specific light absorption metric 176 (Rₙ) may be further based on the phase φₙ at each wavelength λₙ relative to a reference phase (e.g., the phase φₙ at a first wavelengths λ₁):

$${R}_{1} = \frac{{AC}_{{λ}_{1}}}{{DC}_{{λ}_{1}}} \qquad [ Eq . 13 ]$$

$${R}_{2} = \frac{{AC}_{{λ}_{2}}}{{DC}_{{λ}_{2}}} + {α}_{2} cos ( {ϕ}_{2} - {ϕ}_{1} )$$

$${R}_{n} = \frac{{AC}_{{λ}_{n}}}{{DC}_{{λ}_{n}}} + {α}_{n} cos ( - {ϕ}_{1} )$$

where and the phase difference coefficients α₂, . . . , α_{N} (the light absorption coefficients β₀, β₁, . . . β_{N}) are derived from empirical data collected during a hypoxia study as described above.

The blood oxygen saturation (SpO₂) 178 of the user is estimated in step 380.

In some embodiments, the blood oxygen saturation (SpO₂) 178 of the user may be estimated as a function Y of the light absorption metric(s) 176 (e.g., the R-value or any of the improved R′-values described above), the skin tone 175 of the user (e.g., the lightness component L* in the CIELAB color space, an individual typology angle (ITA) quantization value, etc.), and the skin moisture 177 of the user (e.g., the electrical conductivity σ of the skin of the user):

$${SpO}_{2} = Υ ( R , {L}^{*} , σ ) \qquad [ Eq . 14 ]$$

$${SpO}_{2} = Υ ( {R}^{′} , {L}^{*} , σ ) \qquad [ Eq . 15 ]$$

$${SpO}_{2} = Υ ( {R}_{1} , … {R}_{N} , {L}^{*} , σ ) \qquad [ Eq . 16 ]$$

$${SpO}_{2} = Υ ( {R}_{1} , … {R}_{N} , {ϕ}_{1} , … {ϕ}_{N} , {L}^{*} , σ ) \qquad [ Eq . 17 ]$$

In any of those embodiments, the function Y may be derived from empirical data collected during a hypoxia study as described above.

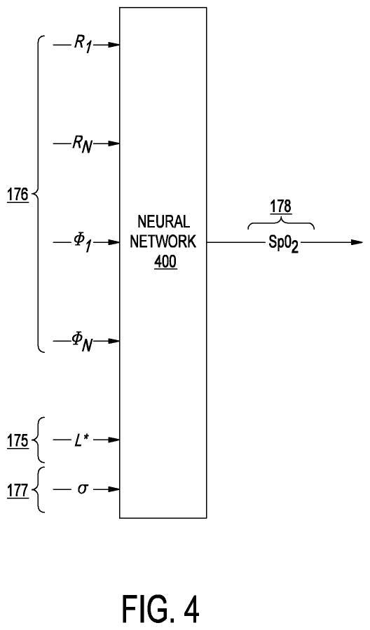

FIG. 4 is a diagram illustrating a neural network 400 trained to learn the multivariate model 174 used for estimating blood oxygen saturation 178 according to exemplary embodiments.

As shown in FIG. 4, a neural network 400 may be trained, using empirical data collected during a hypoxia study, to predict each blood oxygen (SpO₂) measurement 178 of each study participant based on the skin tone 175 of the user (e.g., an individual typology angle (ITA) quantization value, the lightness component L* in the CIELAB color space, etc.), the skin moisture 177 of the user (e.g., the electrical conductivity σ of the skin of the user), and one or more light absorption metrics 176. In some embodiments, the light absorption metrics 176 may include the wavelength-specific light absorption metric Rₙ calculated for each of a plurality of wavelength in as described above. As shown in FIG. 4, the light absorption metrics 176 may also include the phase Øn at each of the wavelengths λₙ. The wavelength-specific light absorption metrics Rₙ (and, in some embodiments, the phases on) may be calculated using the process 300 described above with reference to FIG. 3. In other embodiments, the neural network 400 may be provided with a single light absorption metric 176, for example that is calculated using to Equation 2, Equation 10, or Equation 11 as described above.

Once trained on the empirical data to predict blood oxygen saturation 178 based on skin tone 175, the skin moisture 177, and light absorption metric(s) 176, the weights and biases used by the neural network 400 to predict those predict blood oxygen saturation metrics 178 may form the multivariate model 174 used by the disclosed pulse oximeter 100 to predict the blood oxygen saturation 178 of each user.

Using the modified Goertzel algorithm as described above improves precision relative to existing R-value estimation methods because, unlike the FFT approach or the Goertzel algorithm, the disclosed method is not limited to integer frequency bins. Meanwhile, compared to the FFT approach, the disclosed method provides more accurate magnitude and phase estimations while requiring fewer computational resources.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A wearable health monitoring device for estimating blood oxygen saturation of a user, comprising:
a skin moisture sensor that captures skin moisture data indicative of a skin moisture level of the user;
a skin tone sensor that captures reflectivity data indicative of a skin tone of the user;
a photoplethysmography (PPG) sensor that captures a photoplethysmogram that includes data indicative of light absorption by the skin of the user; and
a hardware processing unit adapted to:
calculate the skin moisture level of the user based on the captured skin moisture data;
quantify the skin tone of the user based on the captured reflectivity data;
calculate one or more light absorption metrics indicative of the light absorption by the skin of the user; and
estimate the blood oxygen saturation of the user by providing the skin moisture level of the user, the skin tone of the user, and the one or more light absorption metrics to a multivariate model generated by a neural network trained on a dataset of blood oxygen saturation values captured from patients having calculated skin moisture levels, quantified skin tones, and calculated light absorption metrics.

2. The device of claim 1, wherein:
the skin moisture sensor comprises a galvanic skin response (GSR) sensor that captures GSR data indicative of the galvanic skin response of the user; and
the hardware processing unit is adapted to calculate the skin moisture level of the user based on the captured GSR data.

3. The device of claim 1, wherein:
the skin moisture sensor comprises a bioimpedance sensor that captures bioimpedance data indicative of a skin impedance of the user; and
the hardware processing unit is adapted to calculate the skin moisture level of the user based on the captured bioimpedance data.

4. The device of claim 3, wherein:
the bioimpedance sensor applies an electrical current via electrodes placed on the skin of the user, measures a voltage drop across the electrodes and an amperage of the electrical current, and calculates a skin impedance level of the user based on the measured voltage drop and the measured amperage; and
the hardware processing unit is adapted to calculate the skin moisture level of the user based on the calculated skin impedance level of the user.

5. The device of claim 1, wherein:
the skin moisture sensor comprises a galvanic skin response (GSR) sensor that captures GSR data indicative of the galvanic skin response of the user and a bioimpedance sensor that captures bioimpedance data indicative of the impedance of the skin of the user; and
the hardware processing unit is adapted to calculate the skin moisture level of the user based on the captured GSR data and the captured bioimpedance data.

6. The device of claim 1, wherein the hardware processing unit is adapted to calculate the one or more light absorption metrics by:
bandpass filtering a PPG signal at each of at least two wavelengths to form a bandpass filtered PPG signal at each of the at least two wavelengths;
estimating a pulsatile frequency of the bandpass filtered PPG signals;
removing a DC component from each bandpass filtered PPG signal to form a zero-mean AC PPG signal at each of the at least two wavelengths;
calculating an AC component of each zero-mean AC PPG signal at a target frequency bin using a modified Goertzel algorithm;
estimating a magnitude of the zero-mean AC PPG signal at each of the at least two wavelengths; and
calculating the one or more light absorption metrics based on the estimated magnitudes and the removed DC components at the at least two wavelengths.

7. The device of claim 6, wherein the target frequency bin is a fractional frequency bin.

8. The device of claim 6, wherein the light absorption metrics comprise a ratio of the estimated magnitude to the removed DC component at each of the at least two wavelengths.

9. The device of claim 8, wherein the light absorption metrics further comprise a phase of each zero-mean AC PPG signal at each of the at least two wavelengths.

10. The device of claim 1, wherein the device is a fingertip sensor or a wearable health monitoring device.

11. A method for estimating blood oxygen saturation of a user, the method comprising:
capturing skin moisture data indicative of a skin moisture level of the user;
capturing reflectivity data indicative of a skin tone of the user;
capturing a photoplethysmogram (PPG) that includes data indicative of light absorption by the skin of the user;
calculating the skin moisture level of the user based on the captured skin moisture data;
quantifying the skin tone of the user based on the captured reflectivity data;
calculating one or more light absorption metrics indicative of the light absorption by the skin of the user; and
estimating the blood oxygen saturation of the user by providing the skin moisture level of the user, the skin tone of the user, and the one or more light absorption metrics to a multivariate model generated by a neural network trained on a dataset of blood oxygen saturation values captured from patients having calculated skin moisture levels, quantified skin tones, and calculated light absorption metrics.

12. The method of claim 11, wherein capturing the skin moisture data comprises capturing galvanic skin response (GSR) data.

13. The method of claim 11, capturing the skin moisture data comprises capturing a skin impedance level of the user.

14. The method of claim 13, wherein the skin impedance level of the user is captured by:
applying an electrical current via electrodes placed on the skin of the user;
measuring a voltage drop across the electrodes and an amperage of the electrical current; and
calculating the skin impedance level of the user based on the measured voltage drop and the measured amperage.

15. The method of claim 11, wherein capturing the skin moisture data comprises capturing galvanic skin response (GSR) data and capturing a skin impedance level of the user.

16. The method of claim 11, wherein calculating the one or more light absorption metrics comprises:
bandpass filtering a PPG signal at each of at least two wavelengths to form a bandpass filtered PPG signal at each of the at least two wavelengths;
estimating a pulsatile frequency of the bandpass filtered PPG signals;
removing a DC component from each bandpass filtered PPG signal to form a zero-mean AC PPG signal at each of the at least two wavelengths;
calculating an AC component of each zero-mean AC PPG signal at a target frequency bin using a modified Goertzel algorithm;
estimating a magnitude of the zero-mean AC PPG signal at each of the at least two wavelengths; and
calculating the one or more light absorption metrics based on the estimated magnitudes and the removed DC components at the at least two wavelengths.

17. The method of claim 16, wherein the target frequency bin is a fractional frequency bin.

18. The method of claim 16, calculating the light absorption metrics comprises:
calculating a ratio of the estimated magnitude to the removed DC component at each of the at least two wavelengths.

19. The method of claim 18, wherein calculating the light absorption metrics further comprises:
calculating a phase of each zero-mean AC PPG signal at each of the at least two wavelengths.

20. A method estimating blood oxygen saturation of a user, the method comprising:
capturing a photoplethysmogram (PPG) signal that includes data indicative of light absorption by the skin of the user;
bandpass filtering the PPG signal at each of at least two wavelengths to form a bandpass filtered PPG signal at each of the at least two wavelengths;
estimating a pulsatile frequency of the bandpass filtered PPG signals;
removing a DC component from each bandpass filtered PPG signal to form a zero-mean AC PPG signal at each of the at least two wavelengths;
calculating an AC component of each zero-mean AC PPG signal at a target frequency bin using a modified Goertzel algorithm;
estimating a magnitude of the zero-mean AC PPG signal at each of the at least two wavelengths; and
calculating one or more light absorption metrics based on the estimated magnitudes and the removed DC components at the at least two wavelengths; and
estimating the blood oxygen saturation of the user by providing one or more light absorption metrics to a mathematical model generated by a neural network trained on a dataset of blood oxygen saturation values captured from patients having calculated light absorption metrics.
